(19) United States
(12) Patent Application Publication
(10) Pub. No.: US 2026/0155223 A1
(43) Pub. Date: Jun. 4, 2026

(54) APPARATUS, NON-TRANSITORY STORAGE MEDIUM, AND METHOD FOR DISPLAYING EVALUATION INDEX BASED ON ABNORMAL SHADOW IN MEDICAL IMAGE

(71) Applicant: FUJIFILM Corporation, Tokyo (JP)

(72) Inventors: Keigo NAKAMURA, Tokyo (JP); Sadato AKAHORI, Tokyo (JP); Yuya HAMAGUCHI, Tokyo (JP)

(73) Assignee: FUJIFILM Corporation, Tokyo (JP)

(21) Appl. No.: 19/455,712

(22) Filed: Jan. 21, 2026

Related U.S. Application Data
(63) Continuation of application No. 18/488,056, filed on Oct. 17, 2023.
Application No. 18/488,056 is a continuation of application No. PCT/JP2022/017411, filed on Apr. 8, 2022.

(30) Foreign Application Priority Data
Apr. 23, 2021 (JP) 2021-073618
Dec. 22, 2021 (JP) 2021-208522

(51) Int. Cl. G16H 15/00; G06F 40/103; G06F 40/169; G06T 7/00; G16H 30/40

(52) U.S. Cl. CPC G16H 15/00; G06F 40/103; G06F 40/169; G06T 7/0016; G16H 30/40; G06T 2207/10081; G06T 2207/20084; G06T 2207/30056; G06T 2207/30096; G06T 2207/30168

(57) ABSTRACT

An apparatus includes one or more processors configured to receive a medical image generated by an imaging apparatus, extract an abnormal shadow present in the medical image, derive an evaluation index by analyzing the abnormal shadow, and display the evaluation index, a type of the abnormal shadow, and text corresponding to the abnormal shadow. The above-described text includes a finding regarding the abnormal shadow, and the above-described finding includes a position of the abnormal shadow, a presence or absence of calcification, and the type of the abnormal shadow.

### CROSS-REFERENCE TO RELATED APPLICATIONS

This application is a continuation application of US application No. Ser. No. 18/488,056 which is filed on Oct. 17, 2023 and is a continuation of International Application No. PCT/JP 2022/017411, filed on Apr. 8, 2022, which claims priority from Japanese Patent Application No. 2021-073618, filed on Apr. 23, 2021 and Japanese Patent Application No. 2021-208522, filed on Dec. 22, 2021. The entire disclosure of each of the above applications is incorporated herein by reference.

### BACKGROUND

### 1. Technical Field

The present disclosure relates to a document creation support apparatus, a document creation support method, and a document creation support program.

### 2. Description of the Related Art

In the related art, there have been proposed technologies for improving the efficiency of creation of a medical document such as an interpretation report by a doctor. For example, JP1995-031591A (JP-H7-031591A) discloses a technology of detecting a type and a position of an abnormality included in a medical image and generating an interpretation report including the detected type and position of the abnormality based on fixed phrases.

In addition, WO2020/209382A discloses a technology of creating a medical document using findings representing features related to abnormal shadows included in a medical image.

### SUMMARY

However, in the technologies disclosed in JP1995-031591A (JP-H7-031591A) and WO2020/209382A, in a case where a medical image includes a plurality of regions of interest such as abnormal shadows, sentences are generated for each of the individual regions of interest, and a plurality of generated sentences are listed. Therefore, in a case where a medical document is created using a plurality of listed sentences, the medical document may not be easy to read. That is, the technologies disclosed in JP1995-031591A (JP-H7-031591A) and WO2020/209382A may not be able to appropriately support the creation of medical documents.

The present disclosure has been made in view of the above circumstances, and an object of the present disclosure is to provide a document creation support apparatus, a document creation support method, and a document creation support program capable of appropriately supporting the creation of a medical document even in a case where a medical image includes a plurality of regions of interest.

According to an aspect of the present disclosure, there is provided an apparatus including one or more processors which are configured to receive a medical image generated by an imaging apparatus, extract an abnormal shadow present in the medical image, derive an evaluation index by analyzing the abnormal shadow, and display the evaluation index, a type of the abnormal shadow, and text corresponding to the abnormal shadow. The text includes a finding regarding the abnormal shadow, and the finding includes a position of the abnormal shadow, a presence or absence of calcification, and the type of the abnormal shadow.

In addition, the one or more processors of the apparatus according to the aspect of the present disclosure are configured to extract a plurality of abnormal shadows and display a plurality of evaluation indexes, types corresponding to the plurality of abnormal shadows, and text corresponding to one of the plurality of abnormal shadows.

In addition, the one or more processors of the apparatus according to the aspect of the present disclosure are configured to change an amount of the finding included in the text based on the evaluation index.

In addition, the one or more processors of the apparatus according to the aspect of the present disclosure are configured to extract the abnormal shadow by using a trained model.

In addition, the one or more processors of the apparatus according to the aspect of the present disclosure are configured to receive a correction to the text by a user.

In addition, the one or more processors of the apparatus according to the aspect of the present disclosure are configured to change the evaluation index according to the type of the abnormal shadow.

In addition, the one or more processors of the apparatus according to the aspect of the present disclosure are configured to derive the evaluation index based on a progress of the abnormal shadow, which is derived from information about a past examination.

In addition, according to an aspect of the present disclosure, there is provided a non-transitory computer-readable storage medium storing a program for causing at least one or more processors provided in an apparatus, to perform a process including receiving a medical image generated by an imaging apparatus, extracting an abnormal shadow present in the medical image; deriving an evaluation index by analyzing the abnormal shadow, and displaying the evaluation index, a type of the abnormal shadow, and text corresponding to the abnormal shadow. The text includes a finding regarding the abnormal shadow, and the finding includes a position of the abnormal shadow, a presence or absence of calcification, and the type of the abnormal shadow.

In addition, the process caused by the non-transitory computer-readable storage medium according to the aspect of the present disclosure further includes extracting a plurality of abnormal shadows and displaying a plurality of evaluation indexes, types corresponding to the plurality of abnormal shadows, and text corresponding to one of the plurality of abnormal shadows.

In addition, the process caused by the non-transitory computer-readable storage medium according to the aspect of the present disclosure further includes changing an amount of the finding included in the text based on the evaluation index.

In addition, the process caused by the non-transitory computer-readable storage medium according to the aspect of the present disclosure further includes extracting the abnormal shadow by using a trained model.

In addition, the process caused by the non-transitory computer-readable storage medium according to the aspect of the present disclosure further includes receiving a correction to the text by a user.

In addition, the process caused by the non-transitory computer-readable storage medium according to the aspect of the present disclosure further includes changing the evaluation index according to the type of the abnormal shadow.

In addition, the process caused by the non-transitory computer-readable storage medium according to the aspect of the present disclosure further includes deriving the evaluation index based on a progress of the abnormal shadow, which is derived from information about a past examination.

In addition, according to an aspect of the present disclosure, there is provided a method executed by at least one or more processors provided in an apparatus. The method includes receiving a medical image generated by an imaging apparatus, extracting an abnormal shadow present in the medical image; deriving an evaluation index by analyzing the abnormal shadow, and displaying the evaluation index, a type of the abnormal shadow, and text corresponding to the abnormal shadow. The text includes a finding regarding the abnormal shadow, and
the finding includes a position of the abnormal shadow, a presence or absence of calcification, and the type of the abnormal shadow.

In addition, the method according to the aspect of the present disclosure further includes extracting a plurality of abnormal shadows; and displaying a plurality of evaluation indexes, types corresponding to the plurality of abnormal shadows, and text corresponding to one of the plurality of abnormal shadows.

In addition, the method according to the aspect of the present disclosure further includes changing an amount of the finding included in the text based on the evaluation index.

In addition, the method according to the aspect of the present disclosure further includes extracting the abnormal shadow by using a trained model.

In addition, the method according to the aspect of the present disclosure further includes receiving a correction to the text by a user.

### BRIEF DESCRIPTION OF THE DRAWINGS

FIG. 1 is a block diagram showing a schematic configuration of a medical information system.

FIG. 2 is a block diagram showing an example of a hardware configuration of a document creation support apparatus.

FIG. 3 is a diagram showing an example of an evaluation value table.

FIG. 4 is a block diagram showing an example of a functional configuration of a document creation support apparatus.

FIG. 5 is a diagram showing an example of text in a sentence format.

FIG. 6 is a diagram showing an example of text in a bullet format.

FIG. 7 is a diagram showing an example of text in a tabular format.

FIG. 8 is a flowchart showing an example of a document creation support process.

FIG. 9 is a diagram showing an example of text in a sentence format.

FIG. 10 is a diagram showing an example of text in a tab format.

FIG. 11 is a diagram showing an example of text in a sentence format according to a modification example.

FIG. 12 is a diagram for describing a process related to correction of an evaluation value.

FIG. 13 is a diagram for describing a process related to correction of an evaluation value.

### DETAILED DESCRIPTION

Hereinafter, form examples for implementing a technology of the present disclosure will be described in detail with reference to the drawings.

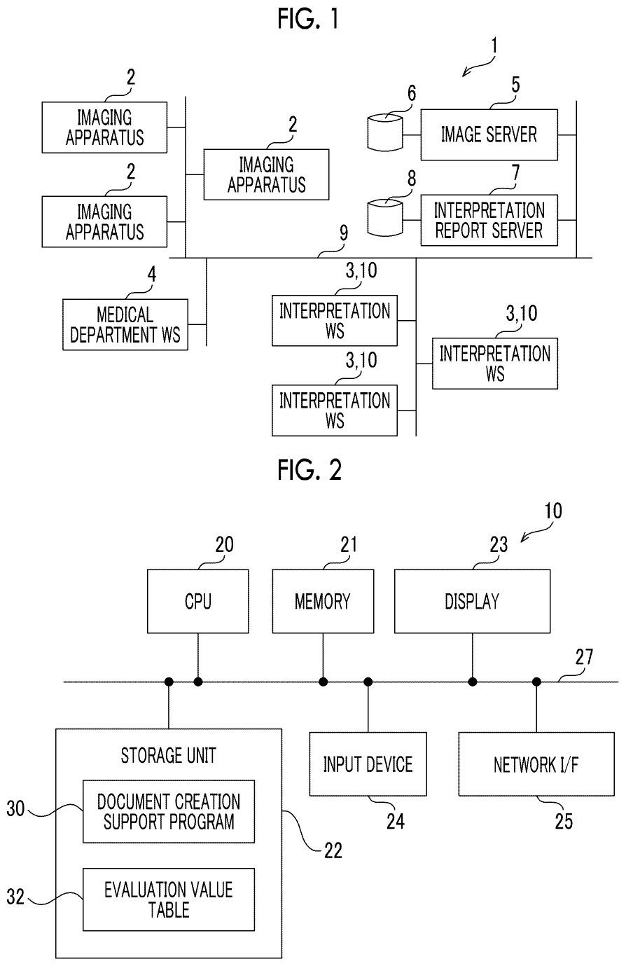

First, a configuration of a medical information system 1 to which a document creation support apparatus according to the disclosed technology is applied will be described with reference to FIG. 1. The medical information system 1 is a system for performing imaging of a diagnosis target part of a subject and storing of a medical image acquired by the imaging based on an examination order from a doctor in a medical department using a known ordering system. In addition, the medical information system 1 is a system for performing interpretation of a medical image and creation of an interpretation report by a radiologist, and viewing the interpretation report and detailed observation of the medical image to be interpreted by a doctor of a medical department that is a request source.

As shown in FIG. 1, the medical information system 1 according to the present embodiment includes a plurality of imaging apparatuses 2, a plurality of interpretation workstations (WS) 3 that are interpretation terminals, a medical department WS 4, an image server 5, an image database (DB) 6, an interpretation report server 7, and an interpretation report DB 8. The imaging apparatus 2, the interpretation WS 3, the medical department WS 4, the image server 5, and the interpretation report server 7 are connected to each other via a wired or wireless network 9 in a communicable state. In addition, the image DB 6 is connected to the image server 5, and the interpretation report DB 8 is connected to the interpretation report server 7.

The imaging apparatus 2 is an apparatus that generates a medical image showing a diagnosis target part of a subject by imaging the diagnosis target part. The imaging apparatus 2 may be, for example, a simple X-ray imaging apparatus, an endoscope apparatus, a computed tomography (CT) apparatus, a magnetic resonance imaging (MRI) apparatus, a positron emission tomography (PET) apparatus, and the like. A medical image generated by the imaging apparatus 2 is transmitted to the image server 5 and is saved therein.

The medical department WS 4 is a computer used by a doctor in the medical department for detailed observation of a medical image, viewing of an interpretation report, creation of an electronic medical record, and the like. In the medical department WS 4, each process such as creating an electronic medical record of a patient, requesting the image server 5 to view an image, and displaying a medical image received from the image server 5 is performed by executing a software program for each process. In addition, in the medical department WS 4, each process such as automatically detecting or highlighting suspected disease regions in the medical image, requesting to view an interpretation report from the interpretation report server 7, and displaying the interpretation report received from the interpretation report server 7 is performed by executing a software program for each process.

The image server 5 incorporates a software program that provides a function of a database management system (DBMS) to a general-purpose computer. In a case where the image server 5 receives a request to register a medical image from the imaging apparatus 2, the image server 5 prepares the medical image in a format for a database and registers the medical image in the image DB 6.

Image data representing the medical image acquired by the imaging apparatus 2 and accessory information attached to the image data are registered in the image DB 6. The accessory information includes information such as an image identification (ID) for identifying individual medical images, a patient ID for identifying a patient who is a subject, an examination ID for identifying examination content, and a unique identification (UID) assigned to each medical image, for example. In addition, the accessory information includes information such as an examination date when a medical image was generated, an examination time, the type of imaging apparatus used in the examination for acquiring the medical image, patient information (for example, a name, an age, and a gender of the patient), an examination part (that is, an imaging part), and imaging information (for example, an imaging protocol, an imaging sequence, an imaging method, imaging conditions, and whether or not a contrast medium is used), and a series number or collection number when a plurality of medical images are acquired in one examination. In addition, in a case where a viewing request from the interpretation WS 3 is received through the network 9, the image server 5 searches for a medical image registered in the image DB 6 and transmits the searched for medical image to the interpretation WS 3 that is a request source.

The interpretation report server 7 incorporates a software program for providing a function of DBMS to a general-purpose computer. In a case where the interpretation report server 7 receives a request to register an interpretation report from the interpretation WS 3, the interpretation report server 7 prepares the interpretation report in a format for a database and registers the interpretation report in the interpretation report database 8. Further, in a case where the request to search for the interpretation report is received, the interpretation report is searched for from the interpretation report DB 8.

In the interpretation report DB 8, for example, an interpretation report is registered in which information, such as an image ID for identifying a medical image to be interpreted, a radiologist ID for identifying an image diagnostician who performed the interpretation, a lesion name, position information of a lesion, findings, and a degree of certainty of the findings, is recorded.

The network 9 is a wired or wireless local area network that connects various apparatuses in a hospital to each other. In a case where the interpretation WS 3 is installed in another hospital or clinic, the network 9 may be configured to connect local area networks of respective hospitals through the Internet or a dedicated line. In any case, it is preferable that the network 9 has a configuration capable of realizing high-speed transmission of medical images such as an optical network.

The interpretation WS 3 requests the image server 5 to view a medical image, performs various types of image processing on the medical image received from the image server 5, displays the medical image, performs an analysis process on the medical image, highlights the medical image based on an analysis result, and creates an interpretation report based on the analysis result. In addition, the interpretation WS 3 supports creation of an interpretation report, requests the interpretation report server 7 to register and view an interpretation report, displays the interpretation report received from the interpretation report server 7, and the like. The interpretation WS 3 performs each of the above processes by executing a software program for each process. The interpretation WS 3 encompasses a document creation support apparatus 10, which will be described later, and in the above processes, processes other than those performed by the document creation support apparatus 10 are performed by a well-known software program, and therefore the detailed description thereof will be omitted here. In addition, processes other than the processes performed by the document creation support apparatus 10 may not be performed in the interpretation WS 3, and a computer that performs the processes may be separately connected to the network 9, and in response to a processing request from the interpretation WS 3, the requested process may be performed by the computer. Hereinafter, the document creation support apparatus 10 encompassed in the interpretation WS 3 will be described in detail.

Next, a hardware configuration of the document creation support apparatus 10 according to the present embodiment will be described with reference to FIG. 2. As shown in FIG. 2, the document creation support apparatus 10 includes a central processing unit (CPU) 20, a memory 21 as a temporary storage area, and a non-volatile storage unit 22. Further, the document creation support apparatus 10 includes a display 23 such as a liquid crystal display, an input device 24 such as a keyboard and a mouse, and a network interface (I/F) 25 connected to the network 9. The CPU 20, the memory 21, the storage unit 22, the display 23, the input device 24, and the network I/F 25 are connected to a bus 27.

The storage unit 22 is realized by a hard disk drive (HDD), a solid state drive (SSD), a flash memory, or the like. A document creation support program 30 is stored in the storage unit 22 as a storage medium. The CPU 20 reads out the document creation support program 30 from the storage unit 22, loads the read document creation support program 30 into the memory 21, and executes the loaded document creation support program 30.

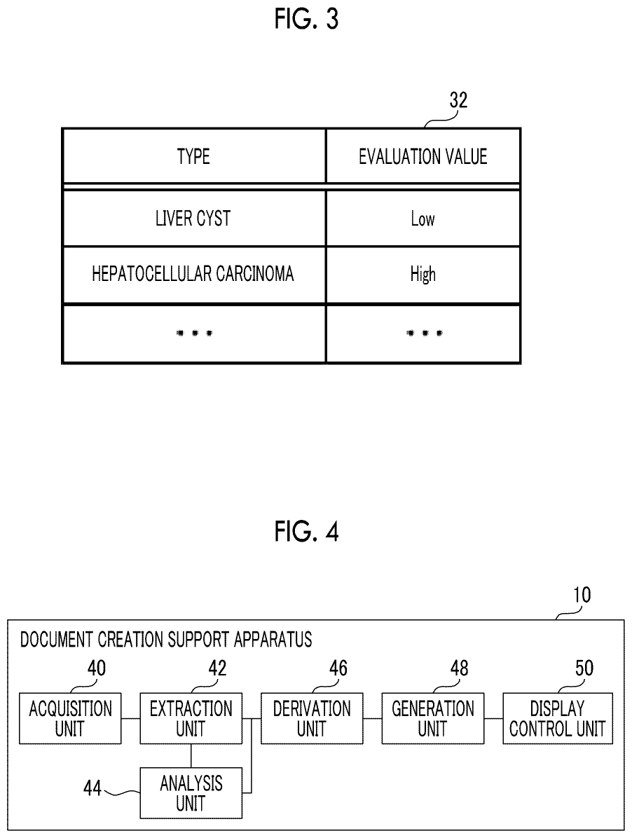

In addition, an evaluation value table 32 is stored in the storage unit 22. FIG. 3 shows an example of the evaluation value table 32. As shown in FIG. 3, in the evaluation value table 32, an evaluation value as a target of the medical document for the abnormal shadow is stored for each type of the abnormal shadow. Examples of medical documents include interpretation reports and the like. In the present embodiment, a larger value is assigned to the evaluation value as the priority described in the interpretation report is higher. FIG. 3 shows an example in which the evaluation value of the hepatocellular carcinoma is a value representing “High” and the evaluation value of the liver cyst is a value representing “Low”. That is, in this example, it is shown that the hepatocellular carcinoma has a higher evaluation value as a target of the interpretation report than the liver cyst. In the example of FIG. 3, the evaluation values are values of two stages of “High” and “Low”, but the evaluation values may be values of three or more stages or continuous values. The above evaluation value is an example of an evaluation index related to the disclosed technology.

In addition, the evaluation value table 32 may be a table in which the degree of severity is associated with each disease name of the abnormal shadow as the evaluation value. In this case, the evaluation value may be, for example, a value that is numerically set for each disease name or an evaluation index such as “MUST” and “WANT”. “MUST” referred to here means that it is always described in the interpretation report, and “WANT” referred to here means that it may or may not be described in the interpretation report. In the example of FIG. 3, the hepatocellular carcinoma is relatively often severe, and the liver cyst is relatively often benign. Therefore, for example, the evaluation value of the hepatocellular carcinoma is set to “MUST”, and the evaluation value of the liver cyst is set to “WANT”.

Next, a functional configuration of the document creation support apparatus 10 according to the present embodiment will be described with reference to FIG. 4. As shown in FIG. 4, the document creation support apparatus 10 includes an acquisition unit 40, an extraction unit 42, an analysis unit 44, a derivation unit 46, a generation unit 48, and a display control unit 50. The CPU 20 executes the document creation support program 30 to function as the acquisition unit 40, the extraction unit 42, the analysis unit 44, the derivation unit 46, the generation unit 48, and the display control unit 50.

The acquisition unit 40 acquires a medical image to be diagnosed (hereinafter referred to as a “diagnosis target image”) from the image server 5 via the network I/F 25. In the following, a case where the diagnosis target image is a CT image of the liver will be described as an example.

The extraction unit 42 extracts a region including an abnormal shadow using a trained model M1 for detecting the abnormal shadow as an example of the region of interest in the diagnosis target image acquired by the acquisition unit 40.

Specifically, the extraction unit 42 extracts a region including an abnormal shadow using a trained model M1 for detecting the abnormal shadow from the diagnosis target image. The abnormal shadow refers to a shadow suspected of having a disease such as a nodule. The trained model M1 is configured by, for example, a convolutional neural network (CNN) that receives a medical image as an input and outputs information about an abnormal shadow included in the medical image. The trained model M1 is, for example, a model trained by machine learning using, as training data, a large number of combinations of a medical image including an abnormal shadow and information specifying a region in the medical image in which the abnormal shadow is present.

The extraction unit 42 inputs the diagnosis target image to the trained model M1. The trained model M1 outputs information specifying a region in which an abnormal shadow included in the input diagnosis target image is present. In addition, the extraction unit 42 may extract a region including an abnormal shadow by a known computer-aided diagnosis (CAD), or may extract a region designated by the user as a region including the abnormal shadow.

The analysis unit 44 analyzes each of the abnormal shadows extracted by the extraction unit 42, and derives findings of the abnormal shadows. Specifically, the extraction unit 42 derives the findings of the abnormal shadow including the type of the abnormal shadow using a trained model M2 for deriving the findings of the abnormal shadow. The trained model M2 is configured by, for example, a CNN that receives, for example, a medical image including an abnormal shadow and information specifying a region in the medical image in which the abnormal shadow is present as inputs, and outputs a finding of the abnormal shadow. The trained model M2 is, for example, a model trained by machine learning using, as training data, a large number of combinations of a medical image including an abnormal shadow, information specifying a region in the medical image in which the abnormal shadow is present, and a finding of the abnormal shadow.

The analysis unit 44 inputs, to the trained model M2, information specifying a diagnosis target image and a region in which the abnormal shadow extracted by the extraction unit 42 for the diagnosis target image is present. The trained model M2 outputs findings of the abnormal shadow included in the input diagnosis target image. Examples of the findings of the abnormal shadow include the position, size, presence or absence of calcification, benign or malignant, presence or absence of irregular margin, type of abnormal shadow, and the like.

The derivation unit 46 acquires information indicating a plurality of abnormal shadows included in the diagnosis target image from the extraction unit 42 and the analysis unit 44. The information indicating the abnormal shadow is, for example, information specifying a region in which the abnormal shadow extracted by the extraction unit 42 is present, and information including findings of the abnormal shadow derived by the analysis unit 44 for the abnormal shadow. In addition, the derivation unit 46 may acquire information indicating a plurality of abnormal shadows included in the diagnosis target image from an external device such as the medical department WS 4. In this case, the extraction unit 42 and the analysis unit 44 are provided by the external device.

Then, the derivation unit 46 derives an evaluation value as the target of the interpretation report for each of the plurality of abnormal shadows represented by the acquired information. The derivation unit 46 derives an evaluation value of the abnormal shadow according to the type of the abnormal shadow.

Specifically, the derivation unit 46 refers to the evaluation value table 32 and derives an evaluation value for each of the plurality of abnormal shadows by acquiring an evaluation value associated with the type of the abnormal shadow for each of the plurality of abnormal shadows.

The generation unit 48 generates text including a description regarding at least one of the plurality of abnormal shadows based on the evaluation value derived by the derivation unit 46. In the present embodiment, the generation unit 48 generates text including a comment on findings regarding a plurality of abnormal shadows in a sentence format. At this time, the generation unit 48 determines the description order of comments on findings of the abnormal shadow to be included in the text according to the evaluation value. Specifically, the generation unit 48 generates text including comments on findings of a plurality of abnormal shadows in order from the abnormal shadow with the highest evaluation value.

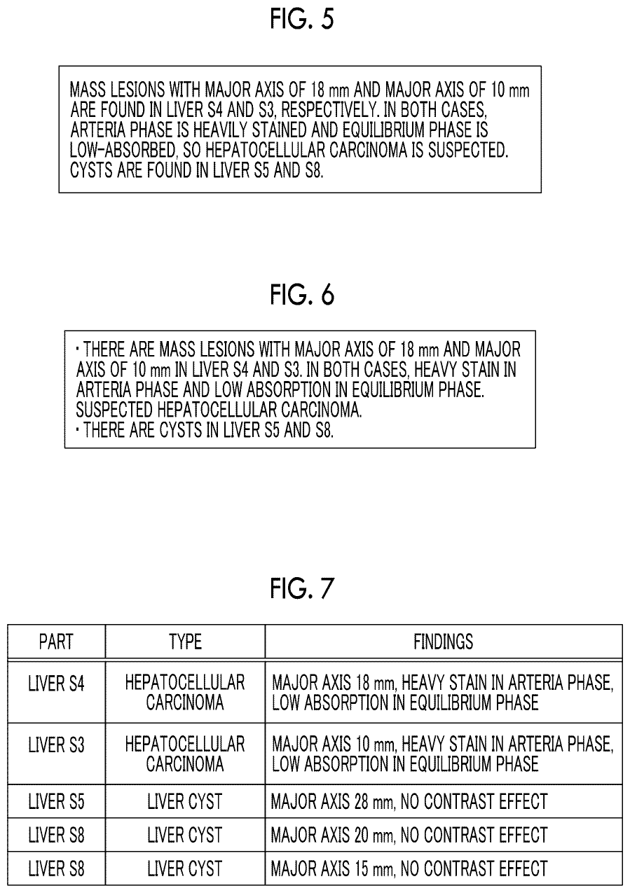

In generating a comment on findings, for example, the generation unit 48 generates the comment on findings by inputting the findings to a recurrent neural network trained to generate text from input words. FIG. 5 shows an example of text including the comments on findings of a plurality of abnormal shadows generated by the generation unit 48. In the example of FIG. 5, text in a sentence format including a comment on findings summarizing findings on two abnormal shadows of the hepatocellular carcinoma and a comment on findings summarizing findings on three abnormal shadows of the liver cyst is shown in order from the abnormal shadow with the highest evaluation value.

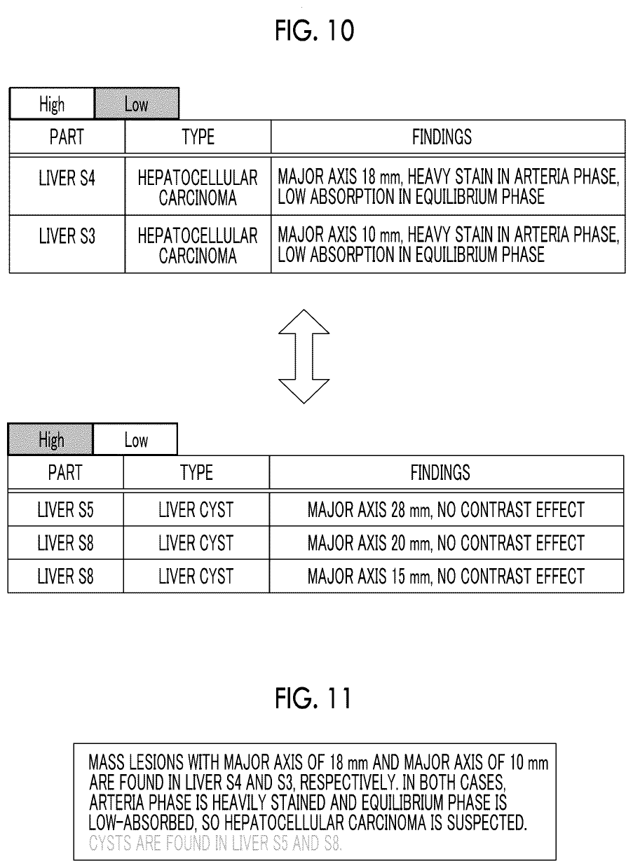

Note that the generation unit 48 may generate text including the description of the plurality of abnormal shadows in a bullet format or in a tabular format. FIG. 6 shows an example of text generated in a bullet format, and FIG. 7 shows an example of text generated in a tabular format. In the example of FIG. 6, similarly to the example of FIG. 5, text in a bullet format including a comment on findings summarizing findings on two abnormal shadows of the hepatocellular carcinoma and a comment on findings summarizing findings on three abnormal shadows of the liver cyst is shown. In the example of FIG. 7, text in a tabular format including findings on each of two abnormal shadows of the hepatocellular carcinoma and findings on each of three abnormal shadows of the liver cyst is shown. In addition, as shown in FIG. 10 as an example, the generation unit 48 may generate text including a description regarding a plurality of abnormal shadows in a tab-switchable format. The upper part of FIG. 10 shows an example in which a tab having an evaluation value of “High” is designated, and the lower part of FIG. 10 shows an example in which a tab having an evaluation value of “Low” is designated.

The display control unit 50 performs control to display the text generated by the generation unit 48 on the display 23. The user corrects the text displayed on the display 23 as necessary and creates an interpretation report.

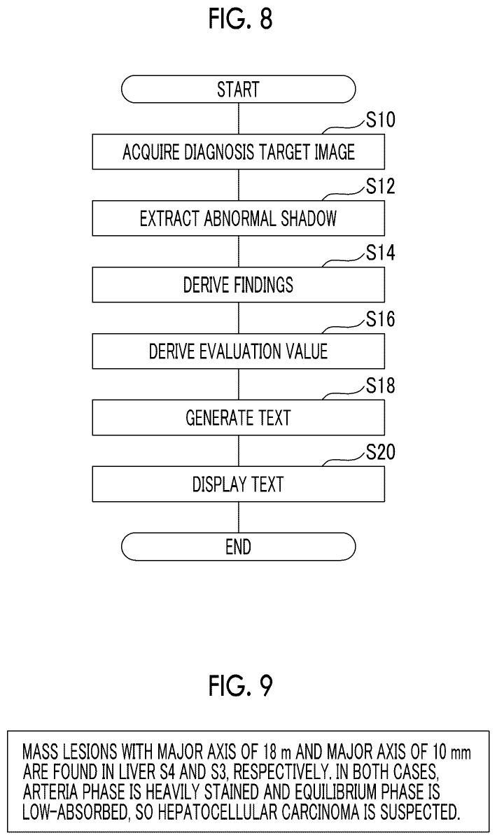

Next, with reference to FIG. 8, operations of the document creation support apparatus 10 according to the present embodiment will be described. The CPU 20 executes the document creation support program 30, whereby a document creation support process shown in FIG. 8 is executed. The document creation support process shown in FIG. 8 is executed, for example, in a case where an instruction to start execution is input by the user.

In Step S10 of FIG. 8, the acquisition unit 40 acquires the diagnosis target image from the image server 5 via the network I/F 25. In Step S12, as described above, the extraction unit 42 extracts regions including abnormal shadows in the diagnosis target image acquired in Step S10 using the trained model M1. In Step S14, as described above, the analysis unit 44 analyzes each of the abnormal shadows extracted in Step S12 using the trained model M2, and derives findings of the abnormal shadows.

In Step S16, as described above, the derivation unit 46 refers to the evaluation value table 32 and derives an evaluation value for each of the plurality of abnormal shadows by acquiring an evaluation value associated with the type of the abnormal shadow derived in Step S14 for each of the plurality of abnormal shadows extracted in Step S12.

In Step S18, as described above, the generation unit 48 generates text including the description regarding the plurality of abnormal shadows extracted in Step S12 based on the evaluation value derived in Step S16. In Step S20, the display control unit 50 performs control to display the text generated in Step S18 on the display 23. In a case where the process of Step S20 ends, the document creation support process ends.

As described above, according to the present embodiment, it is possible to appropriately support the creation of the medical document even in a case where the medical image includes a plurality of regions of interest.

In addition, in the above embodiment, the case where the region of the abnormal shadow is applied as the region of interest has been described, but the present disclosure is not limited thereto. As the region of interest, a region of an organ may be applied, or a region of an anatomical structure may be applied. In a case where a region of an organ is applied as the region of interest, the type of the region of interest means a name of the organ. In addition, in a case where a region of an anatomical structure is applied as the region of interest, the type of the region of interest means a name of the anatomical structure.

In addition, in the above embodiment, the case where the generation unit 48 determines the description order of the comments on findings of the abnormal shadow to be included in the text according to the evaluation value has been described, but the present disclosure is not limited thereto. The generation unit 48 may be configured to determine an abnormal shadow to be included in the text among the plurality of abnormal shadows according to the evaluation value. In this case, a form is exemplified in which the generation unit 48 includes, in the text, only an abnormal shadow whose evaluation value is equal to or greater than a threshold value among the plurality of abnormal shadows. FIG. 9 shows an example of text in this form example. In the example of FIG. 9, text that includes a comment on findings summarizing findings on two abnormal shadows of the hepatocellular carcinoma with an evaluation value of “High” and that does not include a comment on findings on three abnormal shadows of the liver cyst with an evaluation value of “Low” is shown.

In addition, for example, the generation unit 48 may be configured to determine whether or not to include, in the text, a feature of the abnormal shadow to be included in the text according to the evaluation value. In this case, a form is exemplified in which the generation unit 48 includes, in the text, a comment on findings representing the feature for the abnormal shadow whose evaluation value is equal to or greater than the threshold value among the plurality of abnormal shadows. In addition, in this case, a form is exemplified in which the generation unit 48 includes the type of the abnormal shadow for the abnormal shadow whose evaluation value is less than the threshold value among the plurality of abnormal shadows and does not include, in the text, a comment on findings representing the feature of the abnormal shadow. Specifically, as shown in FIGS. 5 and 6, a form is exemplified in which the generation unit 48 includes, in the text, a comment on findings representing a type of the abnormal shadow and a feature of the abnormal shadow for the abnormal shadow of the hepatocellular carcinoma with an evaluation value of “High”, and includes a type of the abnormal shadow in the text and does not include a comment on findings representing a feature of the abnormal shadow in the text for the abnormal shadow of the liver cyst with an evaluation value of “Low”.

In addition, for example, the generation unit 48 may be configured to determine an amount of description of the text according to the evaluation value for the abnormal shadow to be included in the text. In this case, a form is exemplified in which the higher the evaluation value of the abnormal shadow included in the text, the higher an upper limit value of the number of characters of the description regarding the abnormal shadow included in the text is set by the generation unit 48. In addition, for example, the generation unit 48 may generate text including a description regarding the abnormal shadow in order from the abnormal shadow with the highest evaluation value, the text having a predetermined number of characters as an upper limit value. In addition, the upper limit value in this case may be changed by the user by operating a scroll bar or the like.

Further, in a case where the text generated by the generation unit 48 is displayed on the display 23, the display control unit 50 may change a display mode of the description regarding the abnormal shadow included in the text according to the evaluation value. Specifically, as shown in FIG. 11 as an example, the display control unit 50 performs control to display a description regarding an abnormal shadow whose evaluation value is equal to or greater than a threshold value (for example, the evaluation value is “High”) in black characters, and to display a description regarding an abnormal shadow whose evaluation value is less than a threshold value (for example, the evaluation value is “Low”) in gray characters that are lighter than black. In a case where the user performs an operation such as a click on the description regarding the abnormal shadow whose evaluation value is less than the threshold value, the display control unit 50 may employ the same display mode as the description regarding the abnormal shadow whose evaluation value is equal to or greater than the threshold value. In addition, the user may be able to integrate the description regarding the abnormal shadow whose evaluation value is equal to or greater than the threshold value by dragging and dropping the description regarding the abnormal shadow whose evaluation value is less than the threshold value.

Further, for example, the display control unit 50 may perform control to display, according to an instruction from the user, a description regarding the abnormal shadow that has not been displayed on the display 23 according to the evaluation value. In addition, in a case where the user manually inputs the text with respect to the displayed text, the display control unit 50 may perform control to display a description similar to the text manually input by the user, from among the descriptions regarding the abnormal shadow whose evaluation value is less than the threshold value.

In addition, for example, the generation unit 48 may correct the evaluation value according to the purpose of an examination of the diagnosis target image. Specifically, the generation unit 48 corrects the evaluation value of the abnormal shadow that matches the purpose of the examination of the diagnosis target image to be high. For example, in a case where the purpose of the examination is “presence or absence of pulmonary emphysema”, the generation unit 48 corrects the evaluation value of abnormal shadows including the pulmonary emphysema to be high. In addition, for example, in a case where the purpose of the examination is “checking the size of the aneurysm”, the generation unit 48 corrects the evaluation value of abnormal shadows including the aneurysm to be high.

In addition, in the above embodiment, the case where the derivation unit 46 derives the evaluation value of the abnormal shadow for each of the plurality of abnormal shadows according to the type of the abnormal shadow has been described, but the present disclosure is not limited thereto. For example, the derivation unit 46 may be configured to derive the evaluation value according to the presence or absence of a change from the same abnormal shadow detected in the past examination. In this case, a from is exemplified in which the derivation unit 46 detects the same abnormal shadow in the medical image captured for the same imaging part of the same subject in the past examination among the abnormal shadows included in the latest diagnosis target image, and makes the evaluation value of the abnormal shadow that has changed from the abnormal shadow included in the past medical image higher than the evaluation value of the abnormal shadow that has not changed. This is useful for follow-up of abnormal shadows detected in past examinations. Changes in the abnormal shadow referred to here include, for example, a change in the size of the abnormal shadow, a change in the degree of progress of the disease, and the like. Further, in this case, in order to ignore the error, the derivation unit 46 may consider that there has been no change for a change equal to or less than a predetermined amount of change.

In addition, for example, the derivation unit 46 may be configured to derive the evaluation value according to whether or not the same abnormal shadow has been detected in the past examination. In this case, a form is exemplified in which the derivation unit 46 makes the evaluation value of the abnormal shadow, of which the same abnormal shadow is not detected in the medical image captured for the same imaging part of the same subject in the past examination among the abnormal shadows included in the latest diagnosis target image, higher than the evaluation value of the abnormal shadow, of which the same abnormal shadow is detected. This is useful for drawing the user's attention to newly appearing abnormal shadows. Further, for example, the derivation unit 46 may set the evaluation value to the highest value for the abnormal shadow that has been reported in the interpretation report in the past.

Further, for example, in displaying the text, the display control unit 50 may perform control to display a description of the abnormal shadow with the evaluation value higher than at the time of detection in the past examination in an identifiable manner from descriptions of other abnormal shadows. Specifically, the display control unit 50 performs control to display a description of the abnormal shadow whose evaluation value at the time of detection in the past examination is less than the threshold value and whose evaluation value in the current examination is equal to or greater than the threshold value in an identifiable manner from descriptions of other abnormal shadows. Examples of the identifiable display in this case include making at least one of a font size or a font color different from each other.

Also, a plurality of evaluation values described above may be combined. The evaluation value in this case is calculated by, for example, the following Equation (1).

$$Evaluation value = V 1 \times V 2 \times V 3 \qquad ( 1 )$$

V1 is, for example, an evaluation value that is numerically set in advance for each type of abnormal shadow in the evaluation value table 32. V2 is, for example, a value indicating the presence or absence of a change from the same abnormal shadow detected in the past examination, and whether or not the same abnormal shadow has been detected in the past examination. For example, V2 is set to “1.0” in a case where the same abnormal shadow has been detected in the past examination and there is a change, to “0.5” in a case where the same abnormal shadow has been detected in the past examination and there is no change, and to “1.0” in a case where the same abnormal shadow has not been detected in the past examination. Further, V3 is set to, for example, “1.0” in a case where an abnormal shadow matches the purpose of the examination of the diagnosis target image and to “0.5” in a case where an abnormal shadow does not match the purpose of the examination of the diagnosis target image.

In addition, in the above embodiment, the document creation support apparatus 10 may present the evaluation value derived by the derivation unit 46 to the user and receive the evaluation value corrected by the user. In this case, the generation unit 48 generates the text using the evaluation value corrected by the user.

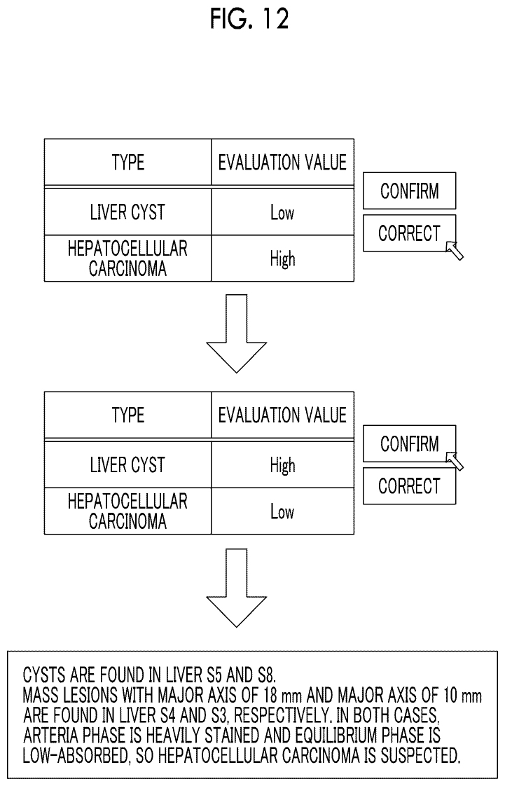

Specifically, as shown in FIG. 12 as an example, the display control unit 50 performs control to display the evaluation value derived by the derivation unit 46 on the display 23. After the user corrects the evaluation value and then performs an operation of confirming the evaluation value, the generation unit 48 generates the text using the evaluation value to which the correction by the user is reflected.

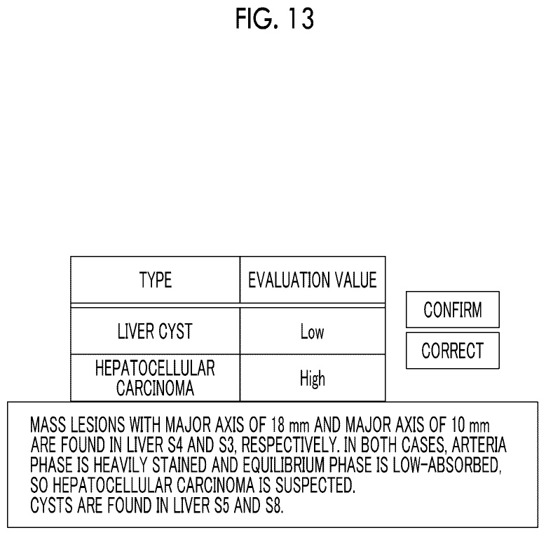

Further, as shown in FIG. 13 as an example, in control in which the text generated by the generation unit 48 is displayed on the display 23, the display control unit 50 may perform control to display the evaluation value derived by the derivation unit 46 together with the text.

In the above embodiment, for example, as hardware structures of processing units that execute various kinds of processing, such as the acquisition unit 40, the extraction unit 42, the analysis unit 44, the derivation unit 46, the generation unit 48, and the display control unit 50, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, there is a form in which a processor for realizing the function of the entire system including a plurality of processing units via one integrated circuit (IC) chip as typified by a system on chip (SoC) or the like is used. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the document creation support program 30 has been described as being stored (installed) in the storage unit 22 in advance; however, the present disclosure is not limited thereto. The document creation support program 30 may be provided in a form recorded in a recording medium such as a compact disc read only memory (CD-ROM), a digital versatile disc read only memory (DVD-ROM), and a universal serial bus (USB) memory. In addition, the document creation support program 30 may be configured to be downloaded from an external device via a network.

The disclosures of Japanese Patent Application No. 2021-073618 filed on Apr. 23, 2021 and Japanese Patent Application No. 2021-208522 filed on Dec. 22, 2021 are incorporated herein by reference in their entirety. In addition, all literatures, patent applications, and technical standards described herein are incorporated by reference to the same extent as if the individual literature, patent applications, and technical standards were specifically and individually stated to be incorporated by reference.

What is claimed is:

1. An apparatus comprising:
one or more processors configured to:
receive a medical image generated by an imaging apparatus;
extract an abnormal shadow present in the medical image;
derive an evaluation index by analyzing the abnormal shadow; and
display the evaluation index, a type of the abnormal shadow, and text corresponding to the abnormal shadow, wherein
the text includes a finding regarding the abnormal shadow, and
the finding includes a position of the abnormal shadow, a presence or absence of calcification, and the type of the abnormal shadow.

2. The apparatus according to claim 1, wherein the one or more processors are configured to
extract a plurality of abnormal shadows; and
display a plurality of evaluation indexes, types corresponding to the plurality of abnormal shadows, and text corresponding to one of the plurality of abnormal shadows.

3. The apparatus according to claim 1, wherein the one or more processor are configured to change an amount of the finding included in the text based on the evaluation index.

4. The apparatus according to claim 1, wherein the one or more processors are configured to extract the abnormal shadow by using a trained model.

5. The apparatus according to claim 1, wherein the one or more processors are configured to receive a correction to the text by a user.

6. The apparatus according to claim 1, wherein the one or more processors are configured to change the evaluation index according to the type of the abnormal shadow.

7. The apparatus according to claim 1, wherein the one or more processors are configured to derive the evaluation index based on a progress of the abnormal shadow, which is derived from information about a past examination.

8. A non-transitory computer-readable storage medium storing a program for causing at least one or more processors provided in an apparatus, to perform a process comprising:
receiving a medical image generated by an imaging apparatus;
extracting an abnormal shadow present in the medical image;
deriving an evaluation index by analyzing the abnormal shadow; and
displaying the evaluation index, a type of the abnormal shadow, and text corresponding to the abnormal shadow, wherein
the text includes a finding regarding the abnormal shadow, and
the finding includes a position of the abnormal shadow, a presence or absence of calcification, and the type of the abnormal shadow.

9. The non-transitory computer-readable storage medium according to claim 8, wherein the process further includes:
extracting a plurality of abnormal shadows; and
displaying a plurality of evaluation indexes, types corresponding to the plurality of abnormal shadows, and text corresponding to one of the plurality of abnormal shadows.

10. The non-transitory computer-readable storage medium according to claim 8, wherein the process further includes changing an amount of the finding included in the text based on the evaluation index.

11. The non-transitory computer-readable storage medium according to claim 8, wherein the process further includes extracting the abnormal shadow by using a trained model.

12. The non-transitory computer-readable storage medium according to claim 8, wherein the process further includes receiving a correction to the text by a user.

13. The non-transitory computer-readable storage medium according to claim 8, wherein the process further includes changing the evaluation index according to the type of the abnormal shadow.

14. The non-transitory computer-readable storage medium according to claim 8, wherein the process further includes deriving the evaluation index based on a progress of the abnormal shadow, which is derived from information about a past examination.

15. A method executed by at least one or more processors provided in an apparatus, the method comprising:
receiving a medical image generated by an imaging apparatus;
extracting an abnormal shadow present in the medical image;
deriving an evaluation index by analyzing the abnormal shadow; and
displaying the evaluation index, a type of the abnormal shadow, and text corresponding to the abnormal shadow, wherein
the text includes a finding regarding the abnormal shadow, and
the finding includes a position of the abnormal shadow, a presence or absence of calcification, and the type of the abnormal shadow.

16. The method according to claim 15 further comprising:
extracting a plurality of abnormal shadows; and
displaying a plurality of evaluation indexes, types corresponding to the plurality of abnormal shadows, and text corresponding to one of the plurality of abnormal shadows.

17. The method according to claim 15 further comprising changing an amount of the finding included in the text based on the evaluation index.

18. The method according to claim 15 further comprising extracting the abnormal shadow by using a trained model.

19. The method according to claim 15 further comprising receiving a correction to the text by a user.

20. The method according to claim 15 further comprising changing the evaluation index according to the type of the abnormal shadow.

21. The method according to claim 15 further comprising deriving the evaluation index based on a progress of the abnormal shadow, which is derived from information about a past examination.
